(19) United States
(12) Patent Application Publication
(10) Pub. No.: US 2019/0185439 A1
(43) Pub. Date: Jun. 20, 2019

(54) RADIOFLUORINATED CARBOXIMIDAMIDES AS IDO TARGETING PET TRACER FOR CANCER IMAGING

(71) Applicant: H. LEE MOFFITT CANCER CENTER & RESEARCH INSTITUTE, INC., Tampa, FL (US)

(72) Inventors: Haibin TIAN, Tampa, FL (US); Robert GILLIES, Tampa, FL (US)

(21) Appl. No.: 16/327,372

(22) PCT Filed: Aug. 24, 2017
(86) PCT No.: PCT/US2017/048370

§ 371 Date: Feb. 22, 2019

Related U.S. Application Data
(60) Provisional application No. 62/378,878, filed on Aug. 24, 2016.

(51) Int. Cl. C07D 271/08; C07B 59/00

(52) U.S. Cl. CPC C07D 271/08; A61K 51/0453; C07B 2200/05; C07B 59/002

(57) ABSTRACT

Radiofluorinated carboximidamides are disclosed as selective IDO enzyme radioligands and generate specific binding in accordance with IDO expression in vitro. MicroPET experiments indicate [¹⁸F]IDO49 specifically accumulate in IDO-expressing tumors which confirmed by Western blot and IHC analysis supported. Using Hela tumor bearing models with IFN-γ treatment confirmed that [¹⁸F]IDO49 accumulation in the IFN-γ treatment tumor mouse. These results can have implications that [¹⁸F]IDO49 has substantial potential as an imaging agent that targets IDO in tumors.

### CROSS REFERENCE TO RELATED APPLICATIONS

This application claims the benefit of priority to U.S. Provisional Application 62/378,878, filed Aug. 24, 2016, which is incorporated by reference herein in its entirety.

### BACKGROUND

The enzyme indoleamine 2,3-dioxygenase (IDO) regulates immune responses through the capacity to degrade the essential amino-acid tryptophan (Trp) into kynurenine (Kyn) and other downstream metabolites that suppress effector T-cell function and favor the differentiation of regulatory T cells. The current experimental evidence indicates that IDO can be expressed by a variety of cell types, including dendritic cells, tumors cells, and stromal cells (Lob, S., et al. (2009). “Inhibitors of indoleamine-2,3-dioxygenase for cancer therapy: can we see the wood for the trees?” Nat Rev Cancer 9(6):445-452). The studies have led to the hypothesis that the IDO pathway is a key regulatory element responsible for induction and maintenance of peripheral immune tolerance in normal physiological situations as well as in pathological conditions including autoimmunity, neuropathology, infection and cancer (Munn, D. H. and A. L. Mellor (2007). “Indoleamine 2,3-dioxygenase and tumor-induced tolerance.” J Clin Invest 117(5):1147-1154). Recent studies have consistently shown expression of IDO in a variety of resected human extra-cerebral tumors, including lung cancer (Astigiano, S., et al. (2005). “Eosinophil granulocytes account for indoleamine 2,3-dioxygenase-mediated immune escape in human non-small cell lung cancer.” Neoplasia 7(4):390-396; Yasui, H., et al. (1986). “Interferon enhances tryptophan metabolism by inducing pulmonary indoleamine 2,3-dioxygenase: its possible occurrence in cancer patients.” Proc Natl Acad Sci USA 83(17):6622-6626), colorectal cancer (Uyttenhove, C., et al. (2003). “Evidence for a tumoral immune resistance mechanism based on tryptophan degradation by indoleamine 2,3-dioxygenase.” Nat Med 9(10):1269-1274; Brandacher, G., et al. (2006). “Prognostic value of indoleamine 2,3-dioxygenase expression in colorectal cancer: effect on tumor-infiltrating T cells.” Clin Cancer Res 12(4):1144-1151), breast cancer (Sakurai, K., et al. (2005). “Study of indoleamine 2,3-dioxygenase expression in patients with breast cancer.” Gan To Kagaku Ryoho 32(11):1546-1549), hepatocellular carcinoma (Ishio, T., et al. (2004) “Immunoactivative role of indoleamine 2,3-dioxygenase in human hepatocellular carcinoma.” J Gastroenterol Hepatol 19(3):319-326), prostate cancer (Uyttenhove, C., et al. (2003). “Evidence for a tumoral immune resistance mechanism based on tryptophan degradation by indoleamine 2,3-dioxygenase.” Nat Med 9(10):1269-1274), pancreatic carcinoma (Id.), and ovarian cancer (Lob, S., et al. (2009). “Inhibitors of indoleamine-2,3-dioxygenase for cancer therapy: can we see the wood for the trees?” Nat Rev Cancer 9(6):445-452). Several of these studies demonstrated that high expression of IDO was associated with a reduced survival (Astigiano, S., et al. (2005). “Eosinophil granulocytes account for indoleamine 2,3-dioxygenase-mediated immune escape in human non-small cell lung cancer.” Neoplasia 7(4):390-396; Brandacher, G., et al. (2006). “Prognostic value of indoleamine 2,3-dioxygenase expression in colorectal cancer: effect on tumor-infiltrating T cells.” Clin Cancer Res 12(4):1144-1151; Okamoto, A., et al. (2005). “Indoleamine 2,3-dioxygenase serves as a marker of poor prognosis in gene expression profiles of serous ovarian cancer cells.” Clin Cancer Res 11(16):6030-6039). These findings indicate that IDO could be a powerful prognostic marker in a variety of tumors, and selective IDO inhibitors for IDO-based therapy and diagnostics are needed.

Currently, there are two methods to monitor IDO1 expression: one is measuring serum concentration ratio of Kyn and Trp and the other is analysis of biopsy samples (Yoshikawa, T., et al. (2010). “Serum concentration of L-kynurenine predicts the clinical outcome of patients with diffuse large B-cell lymphoma treated with R-CHOP.” Eur J Haematol 84(4):304-309). However, serum Kyn/Trp only reflects averaged IDO1 expression but not localized activities. Moreover, other enzymes such as tryptophan 2,3-dioxygenase (TDO) and Indoleamine 2,3-dioxygenase-2 also regulate Trp and Kyn levels because they catalyze the same reaction. Analysis of biopsy samples using immunohistochemistry methods can quantify IDO1 protein expression and RT-PCR can quantify IDO1 mRNA expression, but it is an invasive method and brings significant risk to the patients. Thus, what are needed are new methods and compositions for diagnosing and treating cancers and other disorders that rely on IDO expression and inhibition. New methods and compositions for quantifying IDO expression in vivo and in vitro are also needed. The compositions and methods disclosed herein address these and other needs.

### SUMMARY

In accordance with the purposes of the disclosed materials, compounds, compositions, articles, devices, and methods, as embodied and broadly described herein, the disclosed subject matter relates to compositions and methods of making and using the compositions. In other aspects, the disclosed subject matter relates to compounds having activity as selective IDO inhibitors, methods of making and using the compounds, and compositions and labeled conjugates comprising the compounds. In certain aspects, the disclosed subject matter relates to compounds having the chemical structure shown in Formula I

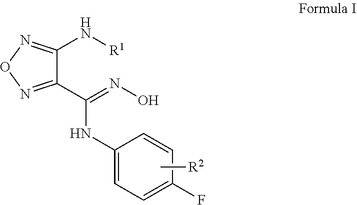

wherein,
R¹ is hydrogen, C₁-C₈ alkyl, C₁-C₈ alkenyl, C₁-C₈ alkynyl, C₁-C₈ haloalkyl, C₁-C₈ haloalkenyl, C₁-C₈ haloalkynyl, C₅-C₆ cycloalkyl, C₅-C₆ heterocycloalkyl, aryl, C₁-C₃ alkylheteroaryl, heteroaryl, C(O)NR⁶R⁷, NHC(O)R⁶, or —O—N═R⁶, any of which is optionally substituted with carbonyl (C═O), carboxyl (—CO₂—), ester (CO₂R⁶), C₁-C₆ alkyl, C₁-C6 alkoxyl, amino, —NR⁶R⁷, —C(O)NR⁶R⁷, C₁-C6 alkylhydroxy, C₅-C₆ cycloalkyl, C₅-C₆ heterocycloalkyl, aryl, heteroaryl, halo, hydroxy, thiol, cyano, nitro, radiolabeled isotope (e.g., ¹⁸F, ¹¹C);
R² is hydrogen, halogen, C₁-C₈ alkyl, C₁-C₈ alkenyl, C₁-C₈ alkynyl, C₁-C₈ haloalkyl, C₁-C₈ haloalkenyl, C₁-C₈ haloalkynyl, C₅-C₆ cycloalkyl, C₅-C₆ heterocycloalkyl, aryl, C₁-C₃ alkylheteroaryl, or heteroaryl, any of which is optionally substituted with carbonyl (C═O), carboxyl (—CO₂—), ester (CO₂R⁶), C₁-C₆ alkyl, C₁-C₆ alkoxyl, amino, —NR⁶R⁷, —C(O)NR⁶R⁷, C₁-C₆ alkylhydroxy, C₅-C₆ cycloalkyl, C₅-C₆ heterocycloalkyl, aryl, heteroaryl, haloaryl, halo, hydroxy, thiol, cyano, nitro, radiolabeled isotope (e.g., ¹⁸F, ¹¹C); and
R⁶ and R⁷ are independently selected from hydrogen, C₁-C₈ alkyl, C₁-C₈ alkenyl, C₁-C₈ alkynyl, C₁-C₈ haloalkyl, C₁-C₈ haloalkenyl, C₁-C₈ haloalkynyl, C₅-C₆ cycloalkyl, C₅-C₆ heterocycloalkyl, aryl, C₁-C₃ alkylheteroaryl, or heteroaryl, any of which is optionally substituted with a halogen;
with the proviso that when R¹ is H, R² is not Cl,
or a pharmaceutically salt thereof.

In still further aspects, the disclosed subject matter relates to methods for treating or diagnosing oncological disorders in a patient. For example, disclosed herein are methods whereby an effective amount of a compound or composition disclosed herein is administered to a patient having an oncological disorder and who is in need of treatment thereof. In another example, disclosed herein are methods whereby an effective amount of a compound or composition disclosed herein is administered to a patient having an oncological disorder and the compound or composition is detected/imaged by a detector. Methods of using the disclosed compounds to inhibit, kill, and/or detect tumor cells, to inhibit IDO, and/or quantify IDO are also disclosed.

Additional advantages of the disclosed subject matter will be set forth in part in the description that follows and the Figures, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 is a graph from an IDO enzyme inhibition assay. Each data point reflects the mean value of n≥3, error bars show standard deviation from the mean.

FIG. 2 is a graph from and IDO HeLa cell assay. IFN-γ induces IDO activity in the HeLa cell lines, the activity of which is inhibited by different IDO inhibitors (IDO49, IDO51, IDO5m).

FIG. 3 is a standard curve of L-Kynurenine's UV absorbance under various concentrations.

FIG. 4 is a pair of radio-TLC profiles of [¹⁸F]IDO5L and [¹⁸F]IDO49 after incubation in physiological saline at 37° C. for 3 h.

FIG. 5 contains representative chromatograms from the Semi-Preparative HPLC separation of the [¹⁸F]IDO49 product.

FIG. 6 contains representative chromatograms from the HPLC analysis of the purified [¹⁸F]IDO49, co-injection with reference IDO49.

FIG. 7A shows data from cell uptake assay of [¹⁸F]IDO at 30 mins, 60 mins and 120 mins FIG. 7B shows data from cell uptake of [¹⁸F]IDO by Hela at 120 mins with the inhibitor of 1-L-MT.

FIG. 8 is a pair of graphs from HeLa cell uptake of [¹⁸F]IDO49 assays (all cells treated by IFN with inhibitor) INCB024360 (IC₅₀=10 nM) NLG919

FIG. 9 shows the PET images of [¹⁸F]IDO49 during 60 min dynamic scan postinjection. For the IFN-γ treatment tumor bearing mice, the tumor radioactivity uptake of [¹⁸F]IDO49 was visualized at 60 min postinjection.

FIG. 10 shows [¹¹C]AMT PET imaging in HeLa Cervical tumor model with IFN-γ treatment.

FIG. 11 shows expression IDO1 of the mice. Injecting mice with IFN-γ (7.5 μg/day. on 1,2,3,4,5,8 days). Euthanasia mice and collect tumor tissues. Extration the protein from the tumor for the western blotting analysis. (“+” represent inject IFN-γ, “−” represent non-inject IFN-γ.

FIGS. 12A through 12F show immunohistochemical expression of IDO. FIG. 12A shows spleen as a negative control in saline treated WT. FIG. 12B shows spleen immunohistochemically stained for the IDO protein as positive control. FIG. 12C shows images at three days of IFN-γ treatment in HeLa tumor tissue. FIG. 12D shows images eight days of IFN-γ treatment in HeLa tumor tissue. FIG. 12E shows thymus from tumor mouse. FIG. 12F shows lymph nodes from tumor mouse.

### DETAILED DESCRIPTION

The materials, compounds, compositions, articles, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “treat” or other forms of the word, such as “treated” or “treatment,” is meant to administer a composition or to perform a method in order to reduce, prevent, inhibit, or eliminate a particular characteristic or event (e.g., tumor growth or survival). The term “control” is used synonymously with the term “treat.”

The term “anticancer” refers to the ability to treat or control cellular proliferation and/or tumor growth at any concentration.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” or “CO” is a short hand notation for C═O, which is also refered to herein as a “carbonyl.”

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —C(O)Z¹ or C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Compounds

Disclosed herein are compounds having Formula I:

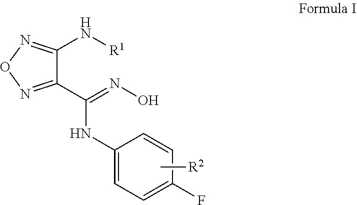

wherein,
R¹ is hydrogen, C₁-C₈ alkyl, C₁-C₈ alkenyl, C₁-C₈ alkynyl, C₁-C₈ haloalkyl, C₁-C₈ haloalkenyl, C₁-C₈ haloalkynyl, C₅-C₆ cycloalkyl, C₅-C₆ heterocycloalkyl, aryl, C₁-C₃ alkylheteroaryl, heteroaryl, C(O)NR⁶R⁷, NHC(O)R⁶, or —O—N═R⁶, any of which is optionally substituted with carbonyl (C═O), carboxyl (—CO₂—), ester (CO₂R⁶), C₁-C₆ alkyl, C₁-C₆ alkoxyl, amino, —NR⁶R⁷, —C(O)NR⁶R⁷, C₁-C₆ alkylhydroxy, C₅-C₆ cycloalkyl, C₅-C₆ heterocycloalkyl, aryl, heteroaryl, halo, hydroxy, thiol, cyano, nitro, or radiolabeled isotope (e.g., ¹⁸F, ¹¹C); and
R² is hydrogen, halogen, C₁-C₈ alkyl, C₁-C₈ alkenyl, C₁-C₈ alkynyl, C₁-C₈ haloalkyl, C₁-C₈ haloalkenyl, C₁-C₈ haloalkynyl, C₅-C₆ cycloalkyl, C₅-C₆ heterocycloalkyl, aryl, C₁-C₃ alkylheteroaryl, or heteroaryl, any of which is optionally substituted with carbonyl (C═O), carboxyl (—CO₂—), ester (CO₂R⁶), C₁-C₆ alkyl, C₁-C₆ alkoxyl, amino, —NR⁶R⁷, —C(O)NR⁶R⁷, C₁-C₆ alkylhydroxy, C₅-C₆ cycloalkyl, C₅-C₆ heterocycloalkyl, aryl, heteroaryl, haloaryl, halo, hydroxy, thiol, cyano, nitro, or radiolabeled isotope (e.g., ¹⁸F, ¹¹C); and
R⁶ and R⁷ are independently selected from hydrogen, C₁-C₈ alkyl, C₁-C₈ alkenyl, C₁-C₈ alkynyl, C₁-C₈ haloalkyl, C₁-C₈ haloalkenyl, C₁-C₈ haloalkynyl, C₅-C₆ cycloalkyl, C₅-C₆ heterocycloalkyl, aryl, C₁-C₃ alkylheteroaryl, or heteroaryl; any of which is optionally substituted with a halogen;
with the proviso that when R¹ is H, R² is not Cl,
or a pharmaceutically salt thereof.

In specific examples, R² is at the meta-position. In specific examples, R² is chlorine at the meta-position.

In specific examples, R¹ and/or R² comprise a radiolabeled isotope. Examples of suitable radiolabeled isotopes are positron-emitting radionuclides. Examples of suitable positron-emitting are carbon-11, nitrogen-13, oxygen-15, fluorine-18, rubidium-82, and strontium-82. Further examples of radiolabeled isotopes are gallium-67, technetium-99m, indium-111, iodine-123, and thallium-201.

In specific examples, R¹ is a C₁-C₈ alkyl substituted with a radiolabeled isotope, e.g., fluorine-18. In further examples, R¹ is C(O)NR⁶R⁷, NHC(O)R⁶, or —O—N═R⁶, wherein R⁶ and R⁷ are independently selected from hydrogen and aryl, heteroaryl, or C₁-C₃ alkylheteroaryl optionally substituted with halogen.

In specific examples, disclosed are compounds having the following formula.

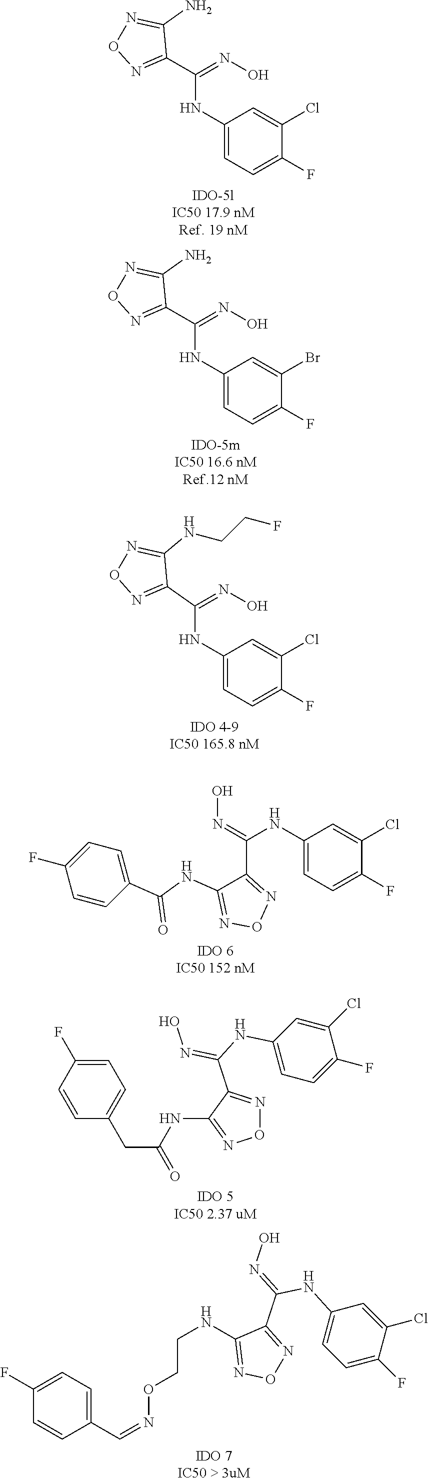 [SMILES: Nc1nonc1/C(=N/O)Nc1ccc(F)c(Cl)c1 ; Nc1nonc1/C(=N/O)Nc1ccc(F)c(Br)c1 ; O/N=C(\Nc1ccc(F)c(Cl)c1)c1nonc1NCCF ; O=C(Nc1nonc1/C(=N/O)Nc1ccc(F)c(Cl)c1)c1ccc(F)cc1 ; O=C(Cc1ccc(F)cc1)Nc1nonc1/C(=N/O)Nc1ccc(F)c(Cl)c1 ; O/N=C(\Nc1ccc(F)c(Cl)c1)c1nonc1NCCO/N=C\c1ccc(F)cc1]

### Methods

Currently, there are no available response biomarkers for 1-D-MT, INCB24360 and Ad.p53 DC vaccine treatment. Hence, it is difficult to monitor the response to this immunotherapy. Thus disclosed herein are methods of monitoring 1-D-MT, INCB24360 and Ad.p53 DC vaccine treatment by administering to a patient undergoing such treatment a compound composition as disclosed herein.

Positron emission tomography (PET) is a powerful molecular imaging tool and allows one to obtain non-invasive, in vivo measurements of multiple molecular processes in various organs using radiolabeled tracers. A PET imaging tracer that is specific for IDO can allow noninvasive detection of IDO levels, which would have many potential applications for variety of cancer detection and staging, and would also provide a new approach to elucidating the role of immunotherapy via IDO in vivo. PET can identify IDO activity in vivo might be useful to predict and monitor IDO-base therapy. One of IDO substrates (α-AMT) have been reported to be useful to identify brain tumors with different profiles of IDO expression (Batista, C. E., et al. (2009). “Imaging correlates of differential expression of indoleamine 2,3-dioxygenase in human brain tumors.” Mol Imaging Biol 11(6):460-466). α-[¹¹C] methyl-L-tryptophan (¹¹C-AMT), an IDO substrate, has been proved to be a good PET tracer for kynurenine pathway (Juhasz, C., et al. (2009). “Quantification of tryptophan transport and metabolism in lung tumors using PET.” J Nucl Med 50(3):356-363). However, IDO1 only involved first step of kynurenine pathway, while increased ¹¹C-AMT uptake by cells is a complicated issue, which involves many enzymes in the tryptophan transportation and metabolism pathway. If the IDO inhibitor can be radiolabeled with fluorine-18 as PET probe to direct measure IDO level in vivo, it can establish the efficacy and potential of IDO probe as a clinically practical for predicting and monitoring vaccine immunotherapy efficacy. Here, the radiofluorinated carboximidamides based on IDO inhibitor INCB024360 as potential IDO targeting tracer for tumor imaging with PET, both [¹⁸F]IDO5L and [¹⁸F]IDO49 were evaluated in vitro and in vivo, including stability, cell occupancy measurements, Western blotting and IDO immunohistochemistry of tumors. First creation of IFN-γ reduced HeLa tumor bearing mouse model was used to explore if [¹⁸F]IDO49 could target IDO as a PET tracer for the imaging of IDO.

Further provided herein are methods of treating or preventing cancer in a subject, comprising administering to the subject an effective amount of a compound or composition as disclosed herein. Additionally, the method can further comprise administering an effective amount of ionizing radiation to the subject.

Methods of killing a tumor cell are also provided herein. The methods comprise contacting a tumor cell with an effective amount of a compound or composition as disclosed herein. The methods can further include administering a second compound or composition (e.g., an anticancer agent) or administering an effective amount of ionizing radiation to the subject.

Also provided herein are methods of radiotherapy of tumors, comprising contacting the tumor with an effective amount of a compound or composition as disclosed herein and irradiating the tumor with an effective amount of ionizing radiation. Methods of treating inflammation in a subject are further provided herein, the methods comprising administering to the subject an effective amount of a compound or composition as described herein. Optionally, the methods can further include administering a second compound or composition (e.g., an anti-inflammatory agent).

The disclosed subject matter also concerns methods for treating a subject having an oncological disorder or condition. In one embodiment, an effective amount of one or more compounds or compositions disclosed herein is administered to a subject having an oncological disorder and who is in need of treatment thereof. The disclosed methods can optionally include identifying a subject who is or can be in need of treatment of an oncological disorder. The subject can be a human or other mammal, such as a primate (monkey, chimpanzee, ape, etc.), dog, cat, cow, pig, or horse, or other animals having an oncological disorder. Means for administering and formulating compounds for administration to a subject are known in the art, examples of which are described herein. Oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lungcancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, or hydrates thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

### EXAMPLES

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

Data are expressed as the mean ±standard deviation. The means were compared using Student's t-test (SPSS 17.0, SPSS, USA). Differences were considered statistically significant at P<0.05.

### Synthesis of Carboximidamides Analogs

The precursors and reference compounds IDO5L, IDO5M were synthesized as previously described (Huang, X., et al. (2015). “Synthesis of [¹⁸F] 4-amino-N-(3-chloro-4-fluorophenyl)-N′-hydroxy-1,2,5-oxadiazole-3-carboximidamide (IDO5L): a novel potential PET probe for imaging of IDO1 expression.” J Labelled Comp Radiopharm 58(4): 156-162). All other chemicals and materials were obtained from commercial sources, were of analytic grade, and were used as received.

The reference compound IDO5L as unlabeled IDO1 inhibitors was synthesized based on the structure of 4-Amino-1,2,5-Oxadiazole-3-Carboximidamide as previouse report (Huang, X., et al. (2015). “Synthesis of [¹⁸F] 4-amino-N-(3-chloro-4-fluorophenyl)-N′-hydroxy-1,2,5-oxadiazole-3-carboximidamide (IDO5L): a novel potential PET probe for imaging of IDO1 expression.” J Labelled Comp Radiopharm 58(4):156-162). The reference compound IDO49 (N-(3-chloro-4-fluorophenyl)-44(2-fluoroethyl)amino)-N′-hydroxy-1,2,5-oxadiazole-3-carboximidamide) and the tosylate precursor 9 (2-((4-(N-(3-chloro-4-fluorophenyl)-N′-hydroxycarbamimidoyl)-1,2,5-oxadiazol-3-yl)aminolethyl 4- methylbenzenesulfonate) were synthesized from compound 7 which is illustrated in Scheme 1.

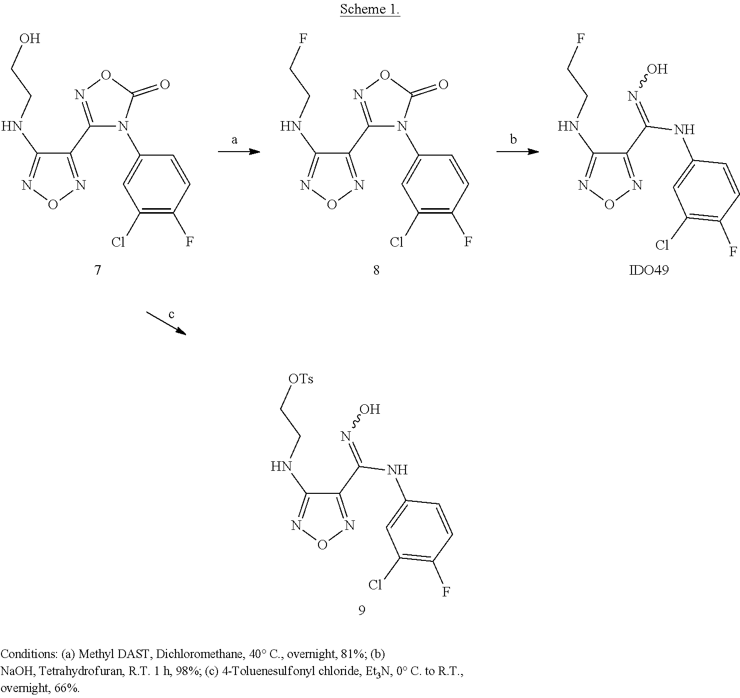

The alcohol 7 was fluorinated by Methyl DAST (Dimethylaminosulfur trifluoride) to give compound 8 in 81% yield. Then the oxadiazolone ring was hydrolyzed under sodium hydroxide to yield amidoxime IDO49 in 98% yield. The tosylate precursor 9 was synthesized by coupling the compound 7 with p-Toluenesulfonyl chloride under base condition in 66% yield. The compound 7 was synthesized from compound 1 using the reported method with minor modification shown in Scheme 2. (Id.; Patent US2010/0015578).

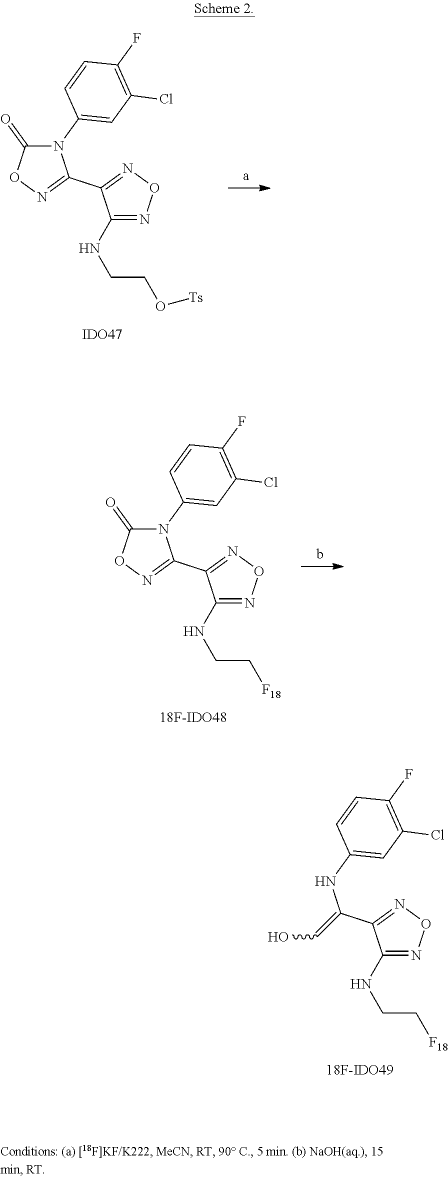

Firstly, the chloro oxime 1 was coupled with amine to yield amidoxime 2 which was converted to amidoxime 3 by overnight refluxed with potassium hydroxide aqueous solution. The amidoxime 3 was then be activated to chloro oxime 4 and followed by coupling with amine to provide compound 5 in 76% 4-step overall yield. The amidoxime of compound 5 was protected as oxadiazolone 6 using 1,1′-carbonyl diimidazole in 94% yield. Finally, the methoxy group was removed by boron tribromide to yield alcohol 7 in 82% yield.

### IDO Enzyme Assay

To investigate the potential influence of IDO ligands carboximidamides analogs on the binding affinity to IDO, some in vitro enzymatic and assays HeLa cellular assay measuring kynurenine formation spectrophotometrically were performed with IDO5L and IDO49.

Human IDO (enzolifesciences ALX-201-333-C050) the oxidative cleavage of the pyrrole ring of the indole nucleus of tryptophan to yield N′-formylkynurenine. The assays were performed at room temperature as described as literature reported with minor revision. (Takikawa, O., et al. (1988). “Mechanism of interferon-gamma action. Characterization of indoleamine 2,3-dioxygenase in cultured human cells induced by interferon-gamma and evaluation of the enzyme-mediated tryptophan degradation in its anticellular activity.” J Biol Chem 263(4):2041-2048). In each well of 96 well-plate, 10 μL of Human IDO (0.05 mg/mL in KHPO₄, 50 mM, PH 6.5) was added into 39 μL buffers (KHPO₄, 50 mM, PH 6.5) and 1 μL inhibitor buffer in DMSO (2000, 600, 200, 60, 20, 6, 2, 0.6, 0.2 μM). Then, 50 μL the substrate buffer (4 mM L-tryptophan, 40 mM ascorbate, 20 μM methylene blue, 0.2 mg/mL catalase) was mixed into each well and incubated at 37° C. for 2 hours. Then, 10 μL of 6.1N trichloroacetic acid was mixed into each well and incubated at 52° C. for 30 min to hydrolyze N-formylkynurenine produced by IDO to kynurenine. The reaction mixture was incubated with 100 μL of 0.02 g/mL p-(dimethylamino)benzaldehyde in acetic acid at room temperature for 10 minutes. The yellow color derived from kynurenine was measured at 492 nm using microplate reader. L-Kynurenine used as the standard, was prepared in a series of concentrations (1000, 500, 200, 100, 50, 20, 10 μM). The percent inhibition at individual concentrations was determined and the average values of duplicates were obtained. The data was processed using nonlinear regression to generate IC₅₀ values (Prism Graphpad).

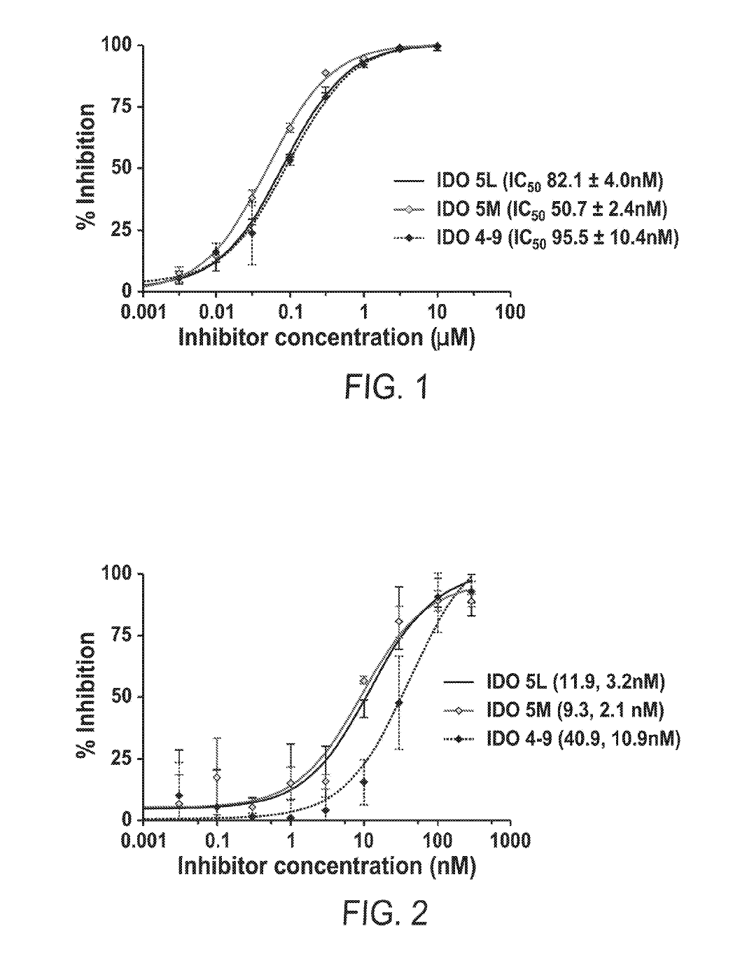

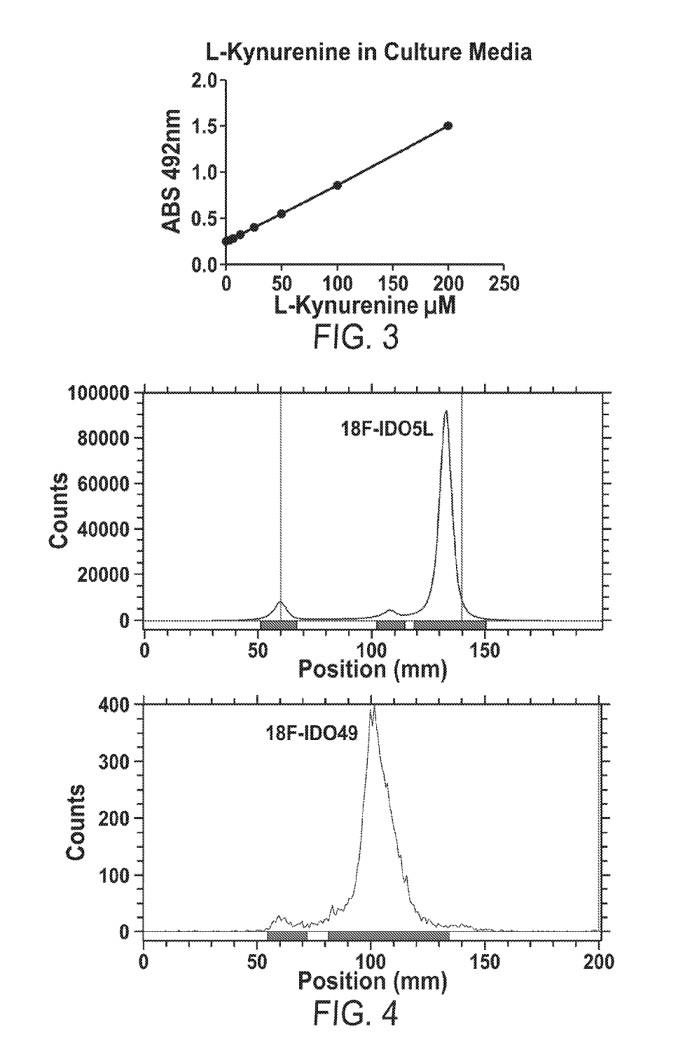

The data that show the amount of enzyme activity remaining at three different inhibitor concentrations are shown in FIGS. 1, 2, and 3. These results suggest a higher affinity of human IDO for IDO5L and IDO49, IDO5L were significantly more potent than the IDO49 and other IDO5m.

### IDO Hela Cells Assay

Hela cells assay was carried out according to the reference (Takikawa, O., et al. (1988). “Mechanism of interferon-gamma action. Characterization of indoleamine 2,3-dioxygenase in cultured human cells induced by interferon-gamma and evaluation of the enzyme-mediated tryptophan degradation in its anticellular activity.” J Biol Chem 263(4):2041-2048; Yue, E. W., et al. (2009). “Discovery of potent competitive inhibitors of indoleamine 2,3-dioxygenase with in vivo pharmacodynamic activity and efficacy in a mouse melanoma model.” J Med Chem 52(23): 7364-7367) with minor modification. HeLa cells were routinely maintained in Earle's Minimum Essential Medium (EMEM) (ATCC 30-2003) and 10% fetal bovine serum. Cells were kept at 37° C. in a humidified incubator supplied with 5% CO₂. The assay was performed as follows: HeLa cells were seeded in a 96 well culture plate at a density of 5×10³ per well in 100 μL culture media. After grown overnight (16 h), human IFN-γ (50 ng/mL final concentration) and serial dilutions of compounds in 100 μL culture medium per well were added into cells. After an additional 48 hours of incubation, 140 μL of the supernatant per well was transferred to a new 96 well plate. Ten microliters of 6.1 N trichloroacetic acid were mixed into each well and incubated at 50° C. for 30 min to hydrolyze N-formylkynurenine produced by IDO to kynurenine. The reaction mixture was then centrifuged for 10 min at 2500 rpm to remove sediments. One hundred microliters of the supernatant per well were transferred to another 96 well plate and mixed with 100 μL of 2% (w/v) p-dimethylaminobenzaldehyde in acetic acid. The yellow color derived from kynurenine was measured at 492 nm using a SPECTRAmax 250 microplate reader. L-Kynurenine, used as the standard, was prepared in a series of concentrations (200, 100, 50, 24, 12.5, 6.3, 3.2, 1.6 μM) in 100 μL HeLa cell culture media and analyzed in the same procedure. The percent inhibition at individual concentrations was determined and the average values of duplicates were obtained. The data was processed using nonlinear regression to generate IC₅₀ values (Prism Graphpad).

### Radioligand Preparation

[¹⁸F]IDO5L were synthesized as previously described (Huang, X., et al. (2015). “Synthesis of [¹⁸F] 4-amino-N-(3-chloro-4-fluorophenyl)-N′-hydroxy-1,2,5-oxadiazole-3-carboximidamide (IDO5L): a novel potential PET probe for imaging of IDO1 expression.” J Labelled Comp Radiopharm 58(4):156-162), [¹¹C]AMT were synthesized as previously described (Huang, X., et al. (2016). “Design and automated production of ¹¹C-alpha-methyl-1-tryptophan (¹¹C-AMT).” Nucl Med Biol 43(5):303-308). The synthesis of the target tracer [¹⁸F]IDO49 (Scheme 1) was performed by the conventional Kryptofix-mediated nucleophilic ¹⁸F-substitution of tosylated precursor IDO47 followed by NaOH hydrolysis, and the labeling yield was determined by analytical HPLC. Aqueous [¹⁸F]fluoride (40-60 mCi) was trapped on a pre-conditioning QMA cartridge and eluted with a mixture of Kryptofix [2,2,2] (800 μL of a 22.6 mg/mL stock solution in MeCN) and K₂CO₃ stock solution (50 μL of a 84 mg/mL stock solution in water). [¹⁸F]fluoride was dried at 120° C. under a stream of nitrogen by azeotropic distillation with anhydrous acetonitrile (3×0.3 mL) to give the no-carrier-added [K/K222]+¹⁸F-complex as a white semi-solid residue. After cooling to room temperature, a solution of tosylate precursor IDO47 (5.0 mg, 20 μmol) in anhydrous DMSO (0.5 mL) was added into the reaction vial. After heated at 90° C. for 5 min, the mixture turned to yellow color. After cooled to the room temperature, aqueous NaOH (2N 0.1 mL) was added to the reaction mixture to hydrolyze the product. After the reaction mixture stirred in room temperature for 15 min, water (8.0 mL) was added. The aqueous solution was then passed through an activated C18 Sep-Pak plus (Waters Corp). The Sep-Pak was rinsed with water (8.0 mL) and the residue solvent was partly removed by air (20 mL). The product ([¹⁸F]IDO49) was slowly eluted through the column with acetonitrile (1.5 mL). After solvent reduced to ˜0.2 mL by a stream of nitrogen in 100° C., water was added (0.7 mL) and the mixture purified semi-preparative HPLC with the retention time of 20.5-22.8min The collected HPLC fraction (˜4 mL) was diluted with water and then passed through the activated C18 Sep-Pak column. After washed by 4 mL of water, the labeled product was eluted by 1.5 mL acetonitrile and dried under a stream of nitrogen in 100° C. Finally, the residue was dissolved in 0.3 mL physiological saline for animal study.

### Determination of Radiochemical Purity and Specific Radioactivity

Chemical and radiochemical purities were assessed on the same sample by HPLC analysis. Specific activity of the radioligands was calculated from 3 consecutive HPLC analyses (average) and determined by comparing the area of the ultraviolet absorbance peak corresponding to the radiolabeled product on the HPLC chromatogram with a standard curve relating mass to ultraviolet absorbance. [¹⁸F]IDO5L and [¹⁸]IDO49 were analyzed with HPLC, analysis was performed on Agilent 1260 using an in-line UV detector (254 nm) and a NaI crystal flow-count radioactivity detector (Lablogic Flow-RAM detector). The analytical HPLC was performed on an Agilent Eclipse XDB C18 column (5 μm, 4.6×250 mm) with the flow rate 1.0 mL/min using MeCN/0.1% acetic acid in H₂O 50/50, 12/88, or 40/60 as an eluent. Retention times were 11.5 minutes for [¹⁸F]IDO49.

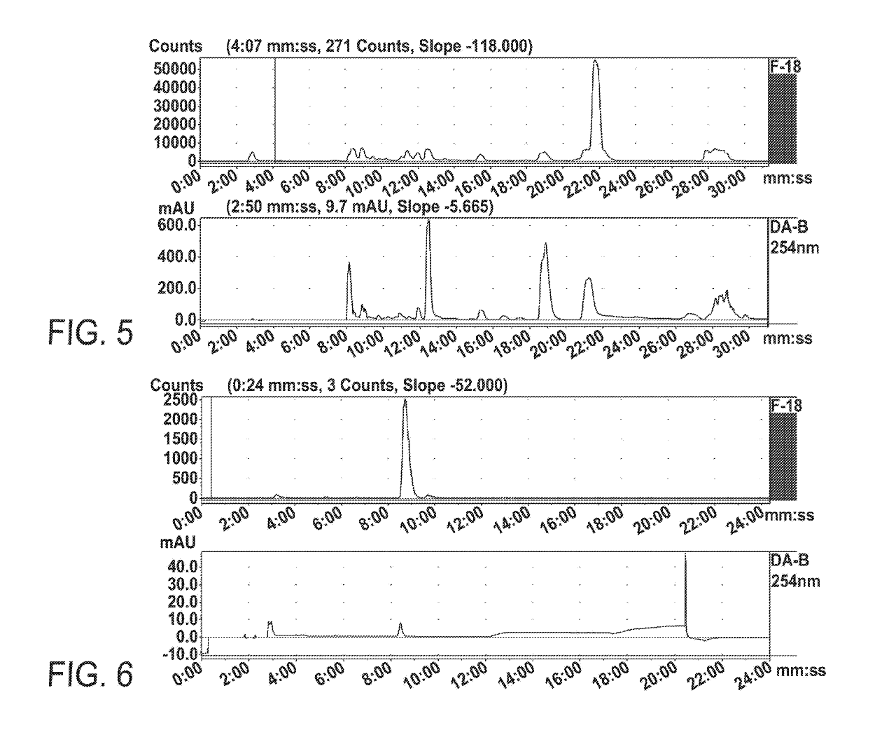

Radiochemical synthesis of [¹⁸F]IDO49 was performed by reacting the tosylate precursor IDO47 with ¹⁸F followed by HPLC purification (t_{R}=20.5-22.8 min min) to afford compound [¹⁸F]IDO49 (FIG. 5). It was isolated in a decay corrected radiochemical yield of 62.8±3.1%, Radiochemical purity (97.5%) and specific activity (1.1 Ci/umol) of the product [¹⁸F]IDO49 were determined by analytical HPLC with the retention time of 8.8 min. The identity of [¹⁸F]IDO49 was confirmed by a co-injection with an nonradioactive standard IDO49 (FIG. 6).

### Lipophilicity Determination

[¹⁸F]IDO49 (2-30 kBq in 30 μL of water) was added to a two-layer system of n-octanol (500 μL) and 0.1M phosphate buffered saline (PBS) pH 7.4 (500 μL) in an eppendorf vial. The vessel was vortexed for 3 min and then centrifuged at 10000 rpm for 2 min. An aliquot of each layer (100 μL) was assessed for radioactivity in a wiper counter. The partition coefficient (logD_{7.4}) was calculated as the decimal logarithm of the ratio between the counted radioactivity in the n-octanol layer and the counted radioactivity in the aqueous layer. logD_{7.4}=(Count of n-octanol sample−Count of background)/(Count of PBS sample−Count of background). CLogP (calculated LogP) values were determined using CHEMBIODRAW ULTRA 12.0 software (Cambridge soft. Perkin-Elmer, Waltham, Mass., USA).

LogD (n-octanol/buffer pH 7.4 partition coefficient) of [¹⁸F]IDO49 was measured using the shake-flask method and the calculated LogP (cLogP, n-octanol/water partition coefficient) was determined using CHEMBIODRAW software. The experiments results showed logD7.4 of [¹⁸F]IDO49 was 1.37±0.02 and clogP was 2.79. Using the same method, the logD of [¹⁸F]IDO5L was 2.13±0.11.

### In Vitro Stability

To examine in vitro stability, approximately 400 μL of [¹⁸F]IDO5L or [¹⁸F]IDO49 and 2 mL of mouse serum at 37° C., were mixed and incubated for 3 h, 200-μL samples were collected at 3 h. The samples were subsequently analyzed Radio-TLC.

The in vitro stability of [¹⁸F]IDO5L and [¹⁸F]IDO49 were evaluated by radio-TLC. As shown in FIG. 4, after incubation in mouse serum at 37° C. for 3 h, >95% of the radioactivity was observed in the form of [¹⁸F]IDO5L and [¹⁸F]IDO49 . This analysis confirmed the absence of radioactive degradation products 3 h in mouse serum at 37° C.

### Hela Cell Uptake of Radioligands

HeLa cells were seeded in a 24 well culture plate at a density of 2×10⁴ per well in 400 μL culture media w/FBS. After grown overnight (16 h), human IFN-γ (25 ng/well, 50 ng/mL final concentration) in 100 μL DMEM w/FBS were added into cells. The cells were rinsed with phosphate-buffered saline (PBS), and 500 μL of Earle's Minimum Essential Medium (EMEM) (ATCC 30-2003) and 10% fetal bovine serum was added to the culture wells. [¹⁸F]IDO5L and [¹⁸F]IDO49 (2 μCi/well) was then added to the wells, and the incubating time was set at 4 time points (15, 30, 60, and 120 minutes) in triplicate. At each given time point, the cell supernatants were discard and wash the cells with PBS (0.5 mL) 3 times, the cells were subsequently lysed with 150 μL of trypsin-EDTA in each well and incubated for 5 min at 37° C., then collect the cell digestion solution and add another 150 μL of PBS into each well to collect the cells. All measurements were performed with a γ-counter (Wizard; PerkinElmer).

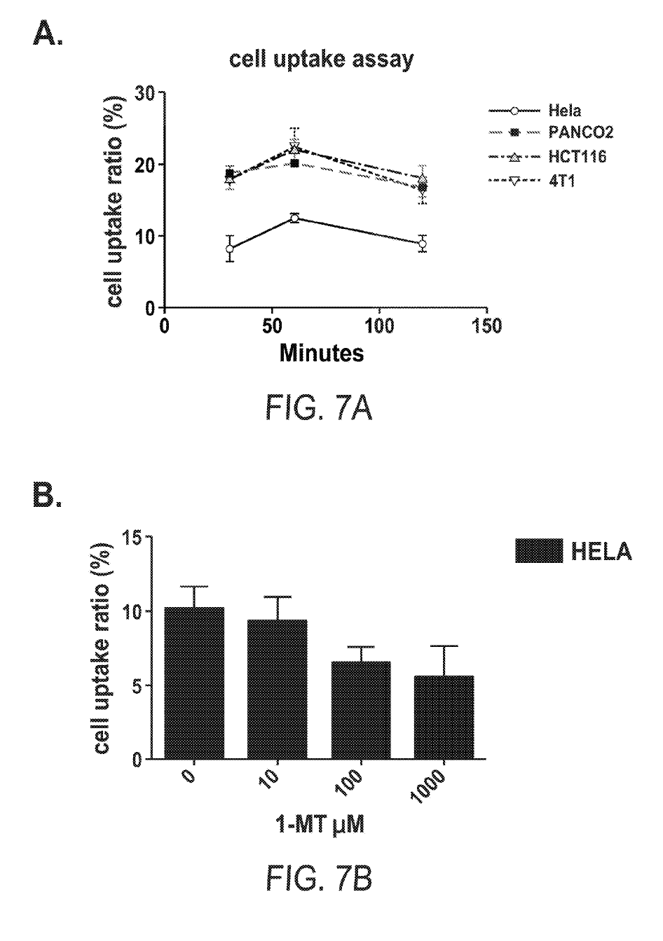

HeLa, PANCO2,HCT116 and 4T1 cells were selected and those cells were then cultured in the absence or presence of IFN-γ as described followed by Western Blotting to confirm IDO expression.

[¹⁸F]IDO5L uptake in Hela, PANCO2, HCT116 and 4T1 cells increased reach maximum in 60 mins, as presented in FIG. 7A, at 30 mins, 60 mins, and 120 mins of incubation. IDO expressed in Hela cells is selectively inhibited by L-1-MT, Cell uptake of [¹⁸F]IDO5L by Hela at 120 mins with the IDO inhibitor of 1-L-MT, L-1-MT showed strong inhibition of IDO activity (FIG. 7B).

Time dependent uptake of [¹⁸F]IDO5L was compared directly with IFN-γ stimulated HeLa cells, PANCO2,HCT116 and 4T1 cells. Uptake of [¹⁸F]IDO5L was remarkably greater than specific activity uptake in HeLa cells in all time points, and matched same inhibitor Hela cell assay in previous report (Yue, E. W., et al. (2009). “Discovery of potent competitive inhibitors of indoleamine 2,3-dioxygenase with in vivo pharmacodynamic activity and efficacy in a mouse melanoma model.” J Med Chem 52(23):7364-7367) suggesting that [¹⁸F]IDO5L is a much better inhibitor for uptake via the IDO expression Hela cell line than other cell lines. Uptake of [¹⁸F]IDO49 in IFN-γ was also evaluated in stimulated HeLa cells with different time points, the results show [¹⁸F]IDO49 was retained at favorable levels in IDO expression Hela cell line with IFN-γ stimulation. A further competitive cell-binding assay was also performed on Hela cells using IDO inhibitors NLG919 and INCB024360 as competitive ligands. When co-incubated with both IDO inhibitors, [¹⁸F]IDO49 uptake in Hela cells decreased in a dose-dependent manner. The cell uptake and competitive cell-binding assay results elucidated the binding of [¹⁸F]IDO49 to IDO.

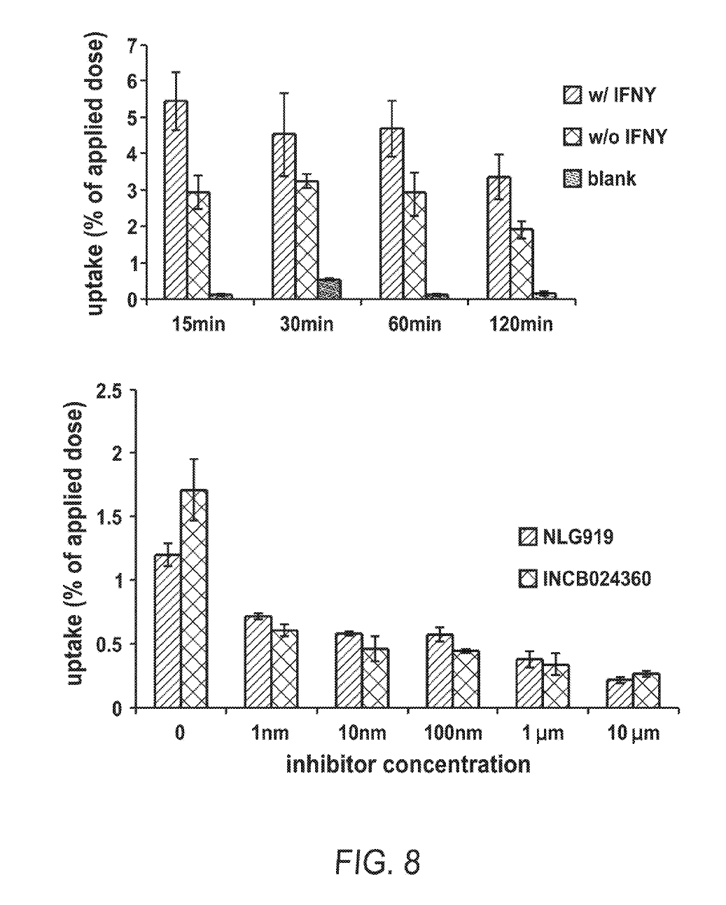

The specific uptake of [¹⁸F]IDO49 in IDO expression IFN-γ stimulated HeLa cells was also confirmed. Nonspecific binding also was determined by blocking with INCB024360 and an alternative competitive IDO inhibitor, NLG919. [¹⁸F]IDO49 was capable of binding IDO with high specific binding in vitro (FIG. 8).

### Competitive Cell-Binding Assay

The binding affinities and specificities of [¹⁸F]IDO5L and [¹⁸F]IDO49 were determined using IDO inhibitors NLG919 and INCB024360 as competitive ligands, HeLa cells preparation procedures is same as above. To each well were added increasing concentrations of NLG919 and INCB024360 (1-10000 nM) in fresh medium (0.5 mL). After incubating for 15 min at 37° C., [¹⁸F]IDO5L and [¹⁸F]IDO49 (2 μCi/well) were then added to the wells, and incubated for 30 minutes separately, the cells were rinsed with PBS (3×0.5 mL) to remove any unbound radioactive materials, the cells were subsequently lysed with 150 μL of trypsin-EDTA in each well and incubated for 5 min at 37° C., then collect the cell digestion solution and add another 150 μL of PBS into each well to collect the cells. All measurements were performed with a γ-counter (Wizard; PerkinElmer). The specificities of radioligands were determined as percentage.

### Tumor Implantation in Mice

All procedures involving animals were reviewed and approved by the Institutional Animal Care and Use Committee (IACUC) at Moffitt Cancer Center. HeLa cells were routinely maintained in Earle's Minimum Essential Medium (EMEM) (ATCC 30-2003) and 10% fetal bovine serum. Cells were kept at 37° C. in a humidified incubator supplied with 5% CO2. The growth media was changed 2 or 3 times per week and the cells were subcultured at a ratio of 1:10 when needed.

Female athymic C57BL/6 nude mice (4-6 weeks old) were ordered from Charles River Laboratories (Stone Ridge, N.Y., U.S.A.) and housed in a temperature and humidity controlled room. After 7 to 14 days of acclimatization, a total of 5×10⁶ Hela cells were inoculated subcutaneously in 200 μL serum-free medium into the right flank of hind leg. Once tumors reach an appropriate size (3-4 mm in diameter) the mice were randomized into 5 groups (4/group). Then the mice in every group were randomized into two subgroups (rHu IFN-γ treatment or no rHu IFN-γ treatment). All the rHu IFN-γ treatment mice were injected i.p. of 1.5×10⁵ U rHu IFN-γ/mouse/day. 24 h later, group one of mice were anesthetized by gas inhalation (2% vol/vol isoflurane in oxygen), then mice were euthanized and tumor tissues were dissected for further IDO western blotting and IHC in vitro. The same operation can be repeated at 48 h, 72 h, 96 h and 120 h points.

### MicroPET Imaging

In order to evaluate the in vivo imaging performance of [¹⁸F]IDO49 to IDO tumors, microPET imaging was performed on nude mice bearing Hela tumor xenografts both rHu IFN-γ treatment and none rHu IFN-γ treatment using the Inveon small-animal PET/CT scanner (Siemens). Anesthesia was induced with 4-5% isoflurane and maintained with 1-3% isofluorane delivered with a mixed air/oxygen through a closed nose cone throughout the PET imaging session. Mice bearing Hela tumor xenografts with rHu IFN-γ treatment were injected with 3.70-7.40 MBq (100-200 μCi) of [¹⁸F]IDO49 in 200 μL of saline, and the baseline control mice with none rHu IFN-γ treatment was injected with the same radiotracer. PET data were collected for 90 min following radioligand administration and reconstructed into 38 dynamic frames of increasing length (6×10, 6×20, 4×30, 9×60, 2×180, 8×300, and 3×600 s). For a comparative study, [¹¹C]AMT PET imaging scan was also performed on Hela tumor mice at 60 min after intravenous injection of [¹¹C]AMT PET (3.7 MBq). The images were reconstructed and the regions of interest (ROIs) were drawn over the tumor and muscle, PET images were converted to percent injected dose per gram (%ID/g of tissue) for evaluation of tumor specificity. Image analysis was performed using the Inevon Research Workplace software.

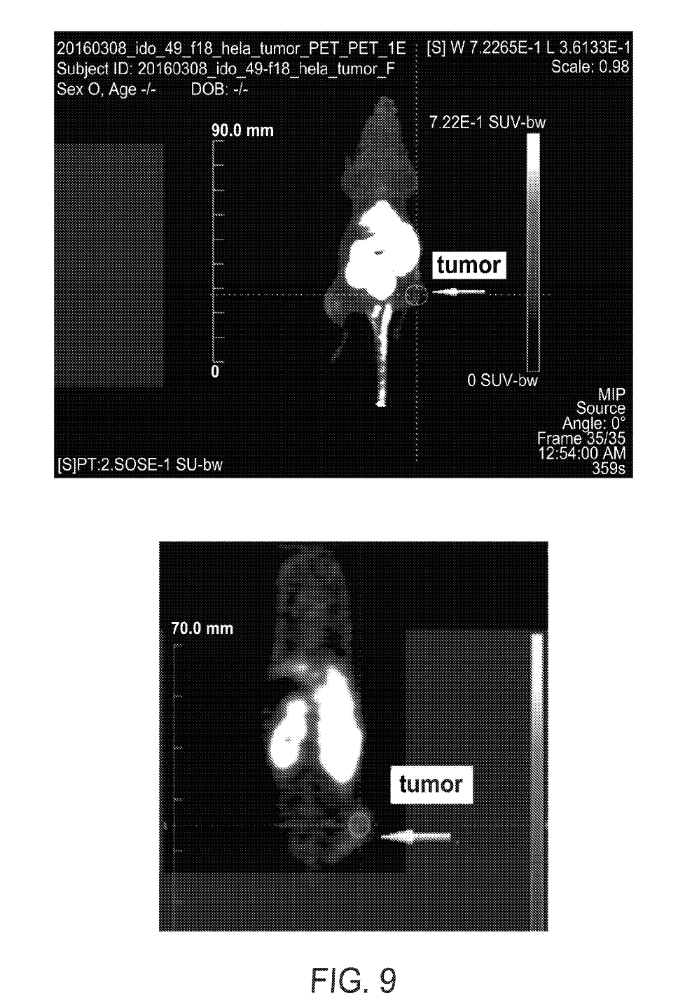

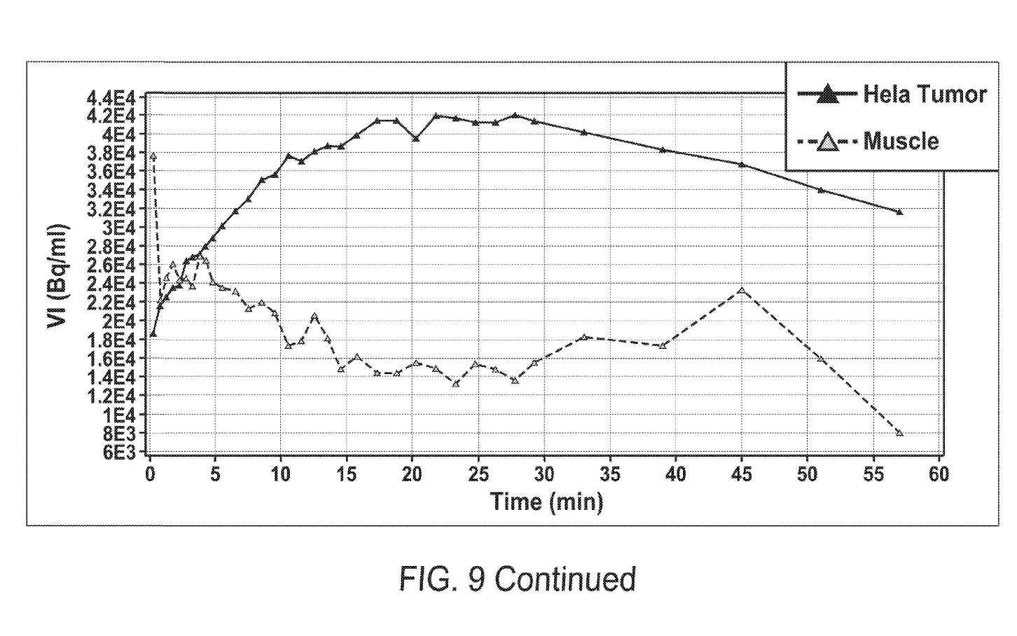

To determine the feasibility of [¹⁸F]IDO49 for noninvasive detection of rHu IFN-γ treatment tumors, whole body PET images for [¹⁸F]IDO49 were obtained in rHu IFN-γ treatment or none treatment mice bearing Hela xenografts. Representative late summed images from 60 min dynamic microPET imaging studies of IFN-γ treatment and none treatment tumor bearing mice administered [¹⁸F]IDO49 were shown in FIG. 9.

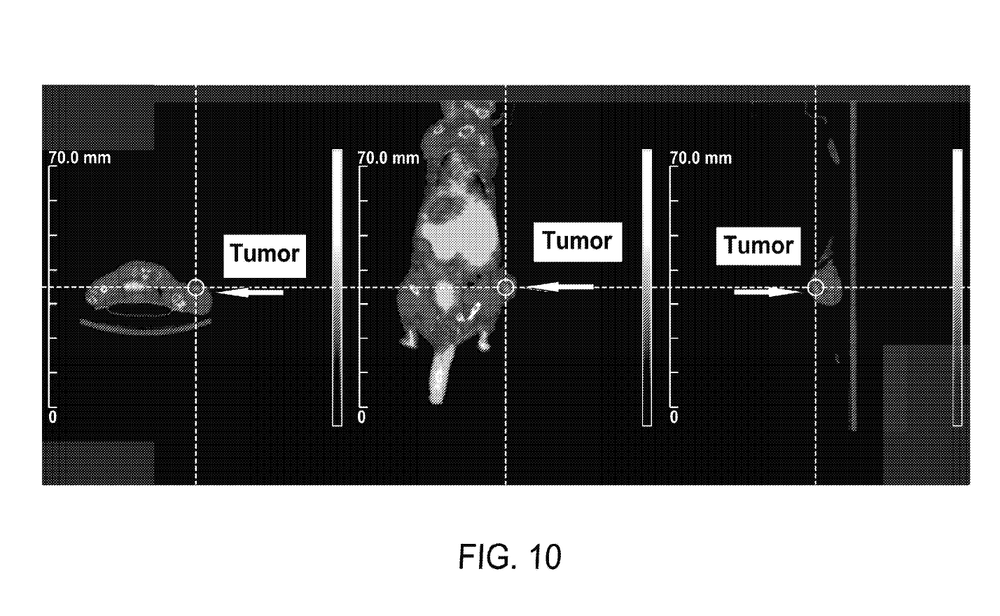

FIG. 9 shows the PET images of [¹⁸F]IDO49 during 60 min dynamic scan postinjection. For the IFN-γ treatment tumor bearing mice, the tumor radioactivity uptake of [¹⁸F]IDO49 was visualized at 60 min postinjection. Radioactive uptake was high in the kidney during 60 min dynamic scan, other radioactivity accumulation was observed in live, which indicates that the majority of the radioligand was cleared from the renal system and live. To confirm tumor uptake of tracer related with induced IDO expression, none IFN-γ treatment tumor bearing mice were evaluated by PET with 60 min dynamic scan. As seen in FIG. 9, none treatment tumor mice had clearly lower activity in the same tumor region compared with the 3 days IFN-γ treatment tumor mice. SUV analysis of the summed images from the dynamic microPET scans confirmed the visual assessment of the images. The average SUV of the tumor with IFN-γ treatment mouse for [¹⁸F]IDO49 was higher than none treatment in tumor mouse. The tumor to muscle (T/M) and relative uptake ratios of [¹⁸F]IDO49 reached the peak (2.29±0.05) at 60 min post-injection. While more IDO specific binding was seen diffusely when [¹⁸C]AMT imaging was performed, PET images with [¹¹C]AMT of IFN-γ treatment tumor mouse shown in FIG. 10, there was significant tracer accumulation were observed in tumor region when compared with same tumor model imaged with [¹¹C]AMT.

### Western Blotting and IDO Immunohistochemistry of Tumors

Tumors were homogenized in the radioimmunoprecipitation assay lysate buffer (consisting of 50 mM Tris-HCl, pH 7.4, 0.1 mM EDTA, 0.1% SDS, 0.15 M NaCl, 1% sodium deoxycholate, and protease inhibitors). After centrifugation at 12,000×g for 20 mM (at 4° C.), supernatants were collected. Protein concentrations were determined using a commercial kit based on the Bradford assay. Bovine serum albumin was used as standard. SDS-polyacrylamide gel electrophoresis (one-dimensional) was performed for separation of the proteins, and the gels were subsequently transferred onto Blot PVDF membranes. The PVDF membranes were blocked in a buffered saline solution (0.05 M Tris-HCl and 0.2 M NaCl, pH 7.4) containing 0.1% (v/v) Tween (TBS with 0.5% bovine serum albumin) and 5% non-fat milk (w/v) for 1 hr at room temperature, and then incubated with the primary antibody (anti-IDO diluted at 1:1000 ; anti-GAPDH antibody diluted at 1:3000; all the primary antibody in TBST containing 1% non-fat milk) for 1 hr at room temperature. The membranes were subsequently rinsed three times (10 mM each) with TBST, incubated with HRP-conjugated anti-mouse IgG or HRP-conjugated anti-rabbit IgG (all the second antibody diluted at 1:10000 and all the primary antibody in TBST containing 1% non-fat milk) respectively, for 1 hr at room temperature, then rinsed three times with TBST (10 mM each). Secondary antibodies on the membranes were detected with an ECL detection system. The densitometry of the detected protein bands was determined using the Scion image analysis software. The densitometry ratio of IDO protein to GAPDH protein was calculated.

Immunohistochemical staining: Tumors were dissected and fixed in 10% (w/v) neutral buffered formalin for 24 hr. Formalin-fixed tissues were processed into paraffin and cut into 5-mm sections on plain slides. Slides were dried for 45 min at 45° C., dewaxed in xylene, and rehydrated through ethanol-graded solutions to water. Slides were stained using a Ventana Discovery XT automated system (Ventana Medical Systems, Tucson, Ariz.) as per manufacturer's protocol with proprietary reagents. Briefly, slides were deparaffinized on the automated system with EZ Prep solution (Ventana). Heat-induced antigen retrieval method was used in Ribo CC (Ventana). The rabbit primary antibody that reacts to IDO, (ab106134, Abcam, Cambridge, Mass.) was used at a 1:25 concentration in Dako antibody diluent (Carpenteria, Calif.) and incubated for 3 hr. The Ventana OmniMap Anti-Rabbit Secondary Antibody was used for 16 mM. The detection system used was the Ventana ChromoMap kit and slides were then counterstained with Hematoxylin. Slides were then dehydrated and coverslipped as per normal laboratory protocol. The immunohistochemical images were taken using an Olympus light microscope equipped with a CCD camera (DP70, Olympus).

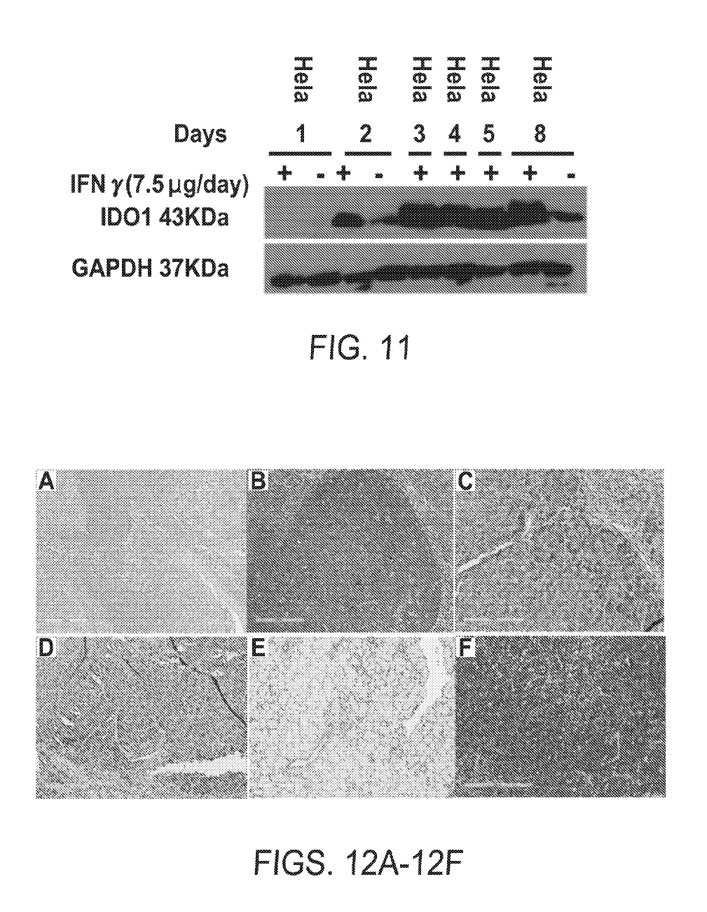

To assess IDO expression in the in vivo tumors, protein levels were evaluated by Western blot. Although there was variation, all tumors with IFN-γ treatment expressed a certain level of IDO except day 1 IFN-γ treatment group (FIG. 11). The histological features of Hela tumor bearing cancer was shown using IDO staining (FIGS. 12A-12F). The levels of IDO in the Hela cancer lesions were estimated by IHC staining. In the IFN-γ-treated tumor mice, the expression levels of IDO were obviously higher in HCCs as compared to the surrounding non-cancerous tissue. The expression levels of IDO in frequently multiple days IFN-γ-treatment were increased compared to sing day IFN-γ-treatment group, whereas the expression levels of IDO were markedly lower in none IFN-γ-treatment group. These findings, together with those of Western blot suggested that induction of IDO might be associated with IFN-γ-treatment in Hela cancer lesions.

The present in vivo study demonstrates for the first time that IDO activity depends on IFN-γ stimulation are regulated in a tissue-specific manner. IFN-γ acts as an antimicrobial agent by activating macrophages, lymphocytes other cells. Some in vitro studies indicate that IFN-γ induced IDO activities are involved in IDO dependent mechanisms by IDO induction, which is mainly induced by IFN-γ. IDO converts L-tryptophan to N-formylkynurenine, and strong IDO induction results in L-tryptophan depletion (Yasui, H., et al. (1986). “Interferon enhances tryptophan metabolism by inducing pulmonary indoleamine 2,3-dioxygenase: its possible occurrence in cancer patients.” Proc Natl Acad Sci USA 83(17): 6622-6626; Murray, H. W., et al. (1989). “Role of tryptophan degradation in respiratory burst-independent antimicrobial activity of gamma interferon-stimulated human macrophages.” Infect Immun 57(3): 845-849; Munn, D. H., et al. (1999). “Inhibition of T cell proliferation by macrophage tryptophan catabolism.” J Exp Med 189(9): 1363-1372).

### Discussion

The distribution of radiolabeled (e.g., radiofluorinated) carboximidamides as imaging tool in tumor-bearing animals is demonstrated herein and shown to determine the specific accumulation in IDO expressing tumors. The uptake of these tracers were assessed in vitro in IDO expression tumor cell lines and also evaluated [¹⁸F]IDO49 in IDO-expressing tumor models using microPET.

Current practice in anti-tumor immune therapy is focused on the regulation by cell-cell communication of checkpoints and checkpoint ligands, such as PD-1 and PD-L1. In fact, tumor-immune interactions are complex and also involve a significant number of soluble factor inhibitors, such as kyneuranines, which are produced by IDO. Although others are working in this area (Liu, X., et al. (2009). “Indoleamine 2,3-dioxygenase, an emerging target for anti-cancer therapy.” Curr Cancer Drug Targets 9(8): 938-952; Aarntzen, E. H., et al. (2013). “In vivo imaging of therapy-induced anti-cancer immune responses in humans.” Cell Mol Life Sci 70(13): 2237-2257), this work is based on the premise that imaging of IDO expression in vivo will inform better decisions during the course of therapy. An enabling aspect of molecular imaging is the ability to capture data longitudinally, i.e. during the course of therapy. As IDO is induced by IFN-γ that is released by activated immune cells, it is likely that the efficacy of IDO inhibition will be critically dependent on timing. An IDO imaging agent could be used during the course of therapy to identify the optimum dosing schedule in individual patients.

Among the newly developed IDO1 inhibitors reported so far (Yue, E. W., et al. (2009). “Discovery of potent competitive inhibitors of indoleamine 2,3-dioxygenase with in vivo pharmacodynamic activity and efficacy in a mouse melanoma model.” J Med Chem 52(23):7364-7367), INCB024360 is an orally bioavailable small molecule inhibitor of IDO1 that has nanomolar potency (IC₅₀=7.1 nM) in both biochemical and cellular assays, potent activity in enhancing T 1 ymphocyte, dendritic cell and natural killer cell responses in vitro, with a high degree of selectivity. The Phase I dose-escalation trial demonstrated that INCB024360 results in greater than 90 percent inhibition of IDO1 activity at generally well-tolerated doses (Liu, X., et al. (2010). “Selective inhibition of IDO1 effectively regulates mediators of antitumor immunity.” Blood 115(17): 3520-3530; Koblish, H. K., et al. (2010). “Hydroxyamidine inhibitors of indoleamine-2,3-dioxygenase potently suppress systemic tryptophan catabolism and the growth of IDO-expressing tumors.” Mol Cancer Ther 9(2):489-498; Gibney, G., et al. (2015). “Updated results from a phase ½ study of epacadostat (INCB024360) in combination with ipilimumab in patients with metastatic melanoma.” Eur J Cancer 51:S106-S107). Moreover, the rapid clearance rate (t1/2<0.5 h) of INCB024360 thus leading to excellent tumor-to-normal tissue contrast, it is preferred for PET imaging agent for less imaging background. Radio-fluorination of carboximidamides as INCB024360 analogs makes them ideal targets for IDO specific PET imaging. In the present study, targeted PET imaging probe were developed for the in vivo detection of IDO level in cervical mouse model.

In order to achieve these objectives, the carboximidamides compounds were synthesized as INCB024360 analogs including reference compounds IDO5m, IDO5L and IDO49, radiolabeling precursor of both IDO5L and IDO49 also were synthesized using two different approaches both easily transposable for the radiosynthesis. The potential influence of IDO5L and IDO49 were investigated on the binding affinity to IDO in vitro. It was observed that both compounds are slightly more potent in the more physiologically relevant HeLa cell-based assays than in the enzyme assays. This may be due to the complexity of the enzyme assay which utilizes a methylene blue ascorbate regeneration system to maintain IDO in the active reduced form or unidentified differences between the recombinant IDO used in the enzyme assay and native IDO in HeLa cells. Nevertheless, a very good correlation between the two assays. The results establish IDO5L as the most potent IDO inhibitor of the series, and its IC₅₀ was determined as 11.9 nM compared with the previously reported IC₅₀ of 19.0 nM (Yue, E. W., et al. (2009). “Discovery of potent competitive inhibitors of indoleamine 2,3-dioxygenase with in vivo pharmacodynamic activity and efficacy in a mouse melanoma model.” J Med Chem 52(23):7364-7367). IDO5L displayed high affinity for IDO with a mean IC₅₀ of 11.9 nM, which is only slightly weaker than the affinity of INCB24360 (7.1 nM), suggesting that attachment of the fluoroethyl moiety has only a minor effect on IDO binding affinity, kinetic analysis demonstrating that three carboximidamides analogs are potent inhibitors of IDO.

Radiochemical synthesis of [¹⁸F]IDO51 was performed in multiple steps as previously reported (Huang, X., et al. (2015). “Synthesis of [(¹⁸)F] 4-amino-N-(3-chloro-4-fluorophenyl)-N′-hydroxy-1,2,5-oxadiazole-3-carboximidamide (IDO5L): a novel potential PET probe for imaging of IDO1 expression.” J Labelled Comp Radiopharm 58(4):156-162), in this study, [¹⁸F]IDO49 was designed with singe step radiolabeling procedure which easy to be separated and have high radiochemical yield. It should be emphasized that the radiofluorinated carboximidamides analogs described here are the first IDO-targeted PET agent to be developed.iny of tryptophan based ¹¹C and ¹⁸F PET agents have already been shown to produce highly defined images of Kynurenine pathway in animal tumor models and human studies (Juhasz, C., et al. (2012). “Tryptophan metabolism in breast cancers: molecular imaging and immunohistochemistry studies.” Nucl Med Biol 39(7):926-932; Guastella, A. R., et al. (2016). “Tryptophan PET Imaging of the Kynurenine Pathway in Patient-Derived Xenograft Models of Glioblastoma.” Mol Imaging 15; Henrottin, J., et al. (2016). “Fully automated radiosynthesis of N(1)-[(¹⁸)F]fluoroethyl-tryptophan and study of its biological activity as a new potential substrate for indoleamine 2,3-dioxygenase PET imaging.” Nucl Med Biol 43(6):379-389; Huang, X., et al. (2016). “Design and automated production of ¹¹C-alpha-methyl-1-tryptophan (¹¹C-AMT).” Nucl Med Biol 43(5): and303-308). Among these substrates of IDO or TDO, displaying of specificity and affinity for IDO expressing in animal or human is still not clear, [¹¹C]AMT can be metabolized by IDO or TDO via the kynurenine pathway, tumoral accumulation of [¹¹C]AMT tracers can occur as a result of tumoral transport, in which high LAT1(L-type amino acid transporter 1) expression may play a central role.

Because it is attractive as a one-step radiolabeling procedure, tosylate precursor was often used by many as a leaving group in the nucleophilic substitution of no-carrier-added [¹⁸F] fluoride (Moussa, I. A., et al. (2011). “Synthesis and in vivo evaluation of [¹⁸F]N-(2-benzofuranylmethyl)-N′-[4-(2-fluoroethoxy)benzyl]piperazine, a novel sigmal receptor PET imaging agent.” Bioorg Med Chem Lett 21(22):6820-6823; Smith, G., et al. (2011). “Radiosynthesis and pre-clinical evaluation of [(¹⁸)F]fluoro-[1,2-(2)H(4)] choline.” Nucl Med Biol 38(1):39-51), it can be used for ¹⁸F-labeling strategy to replace the conventional complex and long process of multiple-step radiolabeling procedure, which shortens reaction time and labor significantly. A new ¹⁸F-labeled, IDO-targeted imaging agent, [¹⁸F]IDO49, was prepared from tosylate precursor IDO47, which allowed rapid and simplified radiolabeling.

In particular, the one-pot, two-step radiosynthesis of [¹⁸G]IDO49 was performed under mild reaction conditions and in radiochemical yields suitable for clinical studies. Furthermore, unlike the radiosyntheses of [¹⁸F]IDO5L, no intermediate purification or coupling steps were required. Only a sample hydrolysis step, final semipreparative purification by HPLC was needed. This synthesis of [¹⁸F]IDO49, is simple and should be able to be readily automated.

High affinity for the imaging target is essential for achieving a target specific signal whereas low lipophilicity is important for promoting adequate tumor entry without encountering adverse effects of high lipophilicity, such slow clearance , high non-specific binding, and lipophilic radiometabolites. [¹⁸F]IDO49 ligands that presented high affinity, better radiochemical yield, and faster or simpler synthesis together with low lipophilicity for fist imaging study were selected.

PET images of Hela tumor bearing models showed [¹⁸F]IDO49 selectively accumulated in tumors with IFN-γ treatment. Indeed, [¹⁸F]IDO49 PET imaging confirmed IFN-γ treatment significantly reduced IDO expression in Hela tumor but not in none IFN-γ treatment control, as IDO is induced by IFN-γ that is released by activated immune cells, these hypothesis were confirmed by the studies of histopathology and IDO IHC of tumors. Since IDO activity has been established as one mechanism by which tumors protect themselves against the host's immune response (Godin-Ethier, J., et al. (2011). “Indoleamine 2,3-dioxygenase expression in human cancers: clinical and immunologic perspectives.” Clin Cancer Res 17(22):6985-6991), here mouse models exposed to IFN-γ were assessed, the studies in vivo demonstrates that IDO activity depends on IFN-γ stimulation are regulated in a tissue-specific manner IFN-γ may play a critical role in activating macrophages, lymphocytes other cells and that IDO expression by IFN-γ functions. Thus, use of Hela tumor bearing models with IFN-γ treatment confirmed that [¹⁸F]IDO49 accumulation in the IFN-γ treatment and tumor mouse was due to in vivo targeting of activated IDO expression rather than nonspecific retention. Some in vitro studies indicate that IFN-γ induced IDO activities are involved in IDO dependent mechanisms, which is mainly induced by IFN-γ. IDO converts L-tryptophan to N-formylkynurenine, and strong IDO induction results in L-tryptophan depletion (Yasui, H., et al. (1986). “Interferon enhances tryptophan metabolism by inducing pulmonary indoleamine 2,3-dioxygenase: its possible occurrence in cancer patients.” Proc Nail Acad Sci USA 83(17):6622-6626; Murray, H. W., et al. (1989). “Role of tryptophan degradation in respiratory burst-independent antimicrobial activity of gamma interferon-stimulated human macrophages.” Infect Immun 57(3):845-849; Munn, D. H., et al. (1999). “Inhibition of T cell proliferation by macrophage tryptophan catabolism.” J Exp Med 189(9):1363-1372). [¹¹C]-AMT PET imaging confirmed that IFN-γ treatment Hela cells primarily express the IDO to convert tryptophan via the kynurenine pathway, furthermore studies will be necessary to assess expression of TDO, LAT1 to clarify correlation of kynurenine pathway. This study is the first time that using IFN-γ treatment tumor model in vivo to identify IDO specificity with PET imaging probe, these radioligands of carboximidamides analogs were characterized, [¹⁸F]IDO49 could access the distribution and intensity of IDO expression as imaging tool.

What is claimed is:

1. A compound having Formula I: 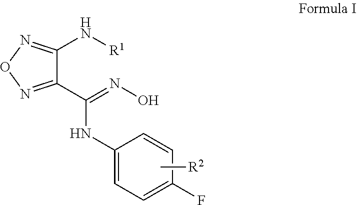
wherein,
R¹ is hydrogen, C₁-C₈ alkyl, C₁-C₈ alkenyl, C₁-C₈ alkynyl, C₁-C₈ haloalkyl, C₁-C₈ haloalkenyl, C₁-C₈ haloalkynyl, C₅-C₆ cycloalkyl, C₅-C₆ heterocycloalkyl, aryl, C₁-C₃ alkylheteroaryl, heteroaryl, C(O)NR⁶R⁷, NHC(O)R⁶, or —O— N═R⁶, any of which is optionally substituted with carbonyl (C═O), carboxyl (—CO₂—), ester (CO₂R⁶), C₁-C₆ alkyl, C₁-C6 alkoxyl, amino, —NR⁶R⁷, —C(O)NR⁶R⁷, C₁-C₆ alkylhydroxy, C₅-C₆ cycloalkyl, C₅-C₆ heterocycloalkyl, aryl, heteroaryl, halo, hydroxy, thiol, cyano, nitro, radiolabeled isotope;
R² is hydrogen, halogen, C₁-C₈ alkyl, C₁-C₈ alkenyl, C₁-C₈ alkynyl, C₁-C₈ haloalkyl, C₁-C₈ haloalkenyl, C₁- C₈ haloalkynyl, C₅-C₆ cycloalkyl, C₅-C₆ heterocycloalkyl, aryl, C₁-C₃ alkylheteroaryl, or heteroaryl, any of which is optionally substituted with carbonyl (C═O), carboxyl (—CO₂—), ester (CO₂R⁶), C₁-C₆ alkyl, C₁-C₆ alkoxyl, amino, —NR⁶R⁷, —C(O)NR⁶R⁷, C₁-C₆ alkylhydroxy, C₅-C₆ cycloalkyl, C₅-C₆ heterocycloalkyl, aryl, heteroaryl, haloaryl, halo, hydroxy, thiol, cyano, nitro, radiolabeled isotope; and
R⁶ and R⁷ are independently selected from hydrogen, C₁-C₈ alkyl, C₁-C₈ alkenyl, C₁-C₈ alkynyl, C₁-C₈ haloalkyl, C₁- C₈ haloalkenyl, C₁-C₈ haloalkynyl, C₅-C₆ cycloalkyl, C₅-C₆ heterocycloalkyl, aryl, C₁-C₃ alkylheteroaryl, or heteroaryl; any of which is optionally substituted with a halogen;
with the proviso that when R¹ is H, R² is not Cl,
or a pharmaceutically salt thereof.

2. The compound of claim 1, wherein R² is at the meta-position.

3. The compound of claim 1, wherein R² is chlorine at the meta-position.

4. The compound of claim 1, wherein R¹ and/or R² is substituted with the radiolabeled isotope.

5. The compound of claim 4, wherein the radiolabeled isotope is a positron-emitting radionuclide.

6. The compound of claim 5, wherein positron-emitting radionuclide comprises carbon-11, nitrogen-13, oxygen-15, fluorine-18, rubidium-82, and strontium-82.

7. The compound of claim 1, wherein R¹ is C₁-C₈ alkyl substituted with a radiolabeled isotope.

8. The compound of claim 7, wherein the radiolabeled isotope is fluorine-18.

9. The compound of claim 1, wherein R¹ is NHC(O)R⁶ or —O—N═R⁶ wherein R⁶ is independently selected from hydrogen and aryl, heteroaryl, or C₁-C₃ alkylheteroaryl optionally substituted with halogen.

10. The compound of claim 1, wherein the compound has one of the following structures:

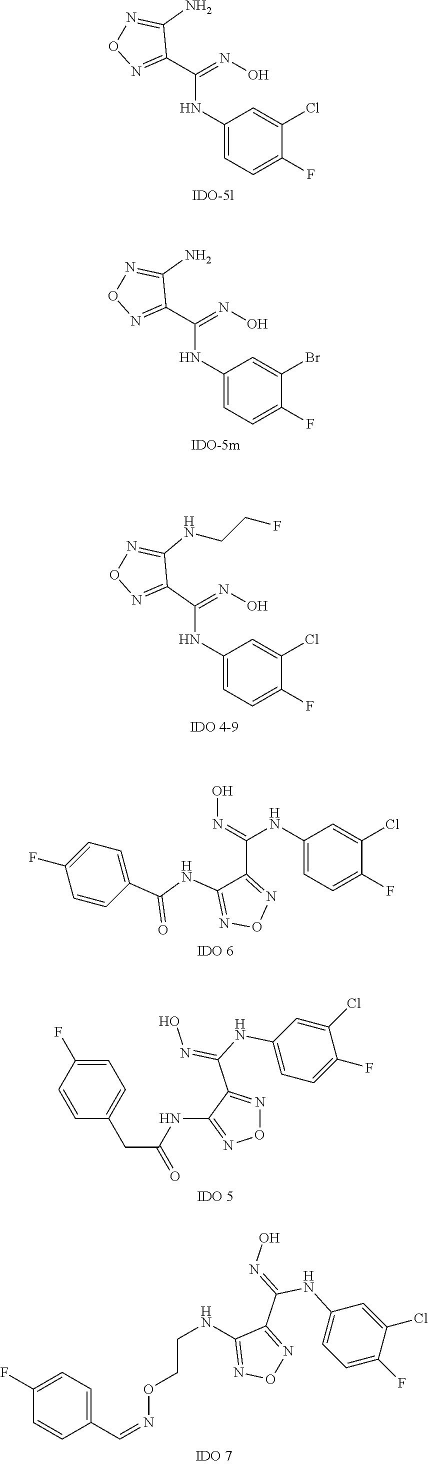 [SMILES: Nc1nonc1/C(=N/O)Nc1ccc(F)c(Cl)c1 ; Nc1nonc1/C(=N/O)Nc1ccc(F)c(Br)c1 ; O/N=C(\Nc1ccc(F)c(Cl)c1)c1nonc1NCCF ; O=C(Nc1nonc1/C(=N/O)Nc1ccc(F)c(Cl)c1)c1ccc(F)cc1 ; O=C(Cc1ccc(F)cc1)Nc1nonc1/C(=N/O)Nc1ccc(F)c(Cl)c1 ; O/N=C(\Nc1ccc(F)c(Cl)c1)c1nonc1NCCO/N=C\c1ccc(F)cc1]

11. A method of quantifying indoleamine 2,3-dioxygenase in a subject, comprising administering to the subject a radiolabeled carboximidamide, and detecting a PET signal from the subject.

12. The method of claim 11, wherein the carboximidamide is the compound of claim 1.

13. The method of claim 11, wherein the carboximidamide is a radiofluorinated carboximidamide.

14. The method of claim 11, wherein the carboximidamide is [¹⁸F]IDO5L or [¹⁸F]IDO49.

15. The method of claim 11, wherein the subject is also administered α-[¹¹C] methyl-L-tryptophan.

16. The method of claim 11, wherein the subject is undergoing vaccine immunotherapy.
